(19) United States
(12) Patent Application Publication
(10) Pub. No.: US 2007/0123976 A1
(43) Pub. Date: May 31, 2007

(54) PSEUDOELASTIC POROUS SHAPE MEMORY MATERIALS FOR BIOMEDICAL AND ENGINEERING APPLICATIONS

(76) Inventors: Bin Yuan, TianHe (CN); Chi Yuen Chung, Hong Kong (HK); Joan Pui Yee Ho, Yuen Long (HK); Min Zhu, TianHe (CN); Kelvin Wai Kwok Yeung, Tai Wnl (HK); Kenneth Man Chee Cheung, Mid-Levois (HK)

Correspondence Address: John W. Boger, Esq.;HESLIN ROTHENBERG FARLEY & MESITI, P.C., 5 Columbia Circle, Albany, NY, 12203, US

(21) Appl. No.: 11/534,275

(22) Filed: Sep. 22, 2006

Related U.S. Application Data
(60) Provisional application No. 60/719,995, filed on Sep. 23, 2005.

(51) Int. Cl. A61F 2/06

(52) U.S. Cl. 623/1.39; 623/11.11; 623/901

(57) ABSTRACT

New porous shape memory materials with the use of different fabrication methods such as hot isostatic pressing technique are provided for biomedical and engineering applications. These new materials have a pseudoelasticity ranging from 0.1% to 50%. The mechanical properties of those materials can be adjusted from 1% to 10%. The pore distribution of these said materials is isotropic and homogenous, and their pore shapes can be tailor-made to be spherical or polygonal as avoiding stress concentration around the pores. The porosity and pore size can be controlled by fabrication process. These materials can exhibit superior pseudoelasticity and mechanical properties during testing than the other porous shape memory alloys fabricated by Self-propagating High-temperature Synthesis (SHS). These advance properties may apply to but not only limited to orthopaedic implants such as artificial bone graft, hip prosthesis and interverbal disc prosthesis; and also for engineering purpose such as damping devices.

### RELATED APPLICATION INFORMATION

This application claims priority from U.S. Provisional Patent Application Ser. No. 60/719,995, filed Sep. 23, 2005. The entire disclosure of Provisional Application Ser. No. 60/719,995 is incorporated herein by reference in its entirety.

### FIELD OF THE INVENTION

This invention relates to shape memory materials, in particular shape memory materials useful for biomedical applications, and methods of forming such materials.

### BACKGROUND OF THE INVENTION AND PRIOR ART

Shape memory materials such as nickel titanium (NiTi) alloys possess excellent shape memory properties, very good mechanical properties, good corrosion resistance and excellent biocompatibility. Porous NiTi alloys are of great interest because the porous structure is likely to enable the exchange of nutrition, and bone and blood vessels in-growth. They are also light in weight. These advantages mean that porous NiTi alloys have great potential in medical applications, especially for orthopaedics such as an artificial bone graft and hip prosthesis that is capable of absorbing impact loading [1-2].

Powder metallurgy (PM) methods are used to prepare porous NiTi alloys by sintering a mixture of elemental Ni and Ti powders. Previously, porous NiTi alloys have been synthesized using many different PM methods, including conventional sintering [3-4], self-propagating high-temperature synthesis (SHS) [5-7] and traditional hot isostatic pressing (HIP) processes [8-9]. Porous NiTi alloys with high porosity and big pore size (about 400-500 μm) have been successfully produced by some of the aforementioned methods. However, the mechanical properties of such porous NiTi SMAs are poor due to anisotropy, non-uniform pore distribution [7], and irregular pore shape [7-9]. This makes such porous NiTi alloys impractical in medical applications.

Ishizaki had succeeded in developing a capsule-free HIP process to make excellent porous ceramic materials [10, 11], which is different from the traditional capsule HIP. Powder compacts are sintered directly under highly pressurized gas. High open porosity can be obtained through this process at high sintering temperature due to the densification of powder compacts being delayed by high-pressure gas. The pore size distribution of the resulting porous ceramic materials is narrower and more symmetric than that of the conventionally sintered porous ceramic materials [12, 13]. Flexural strength [14, 15] and Young's modulus [16] of porous ceramic materials prepared by this HIP process are higher at the same open porosity than those produced by the conventional sintering process.

### SUMMARY OF THE INVENTION

Porous shape memory materials such as porous nickel titanium (NiTi) alloys have great potential in medical applications due to their intrinsic pseudoelasticity. They are also biocompatible to human tissues. However, the pseudoelasticity of the porous NiTi alloys fabricated by the conventional methods such as conventional sintering, self-propagating high-temperature synthesis (SHS) and traditional hot isostatic pressing (HIP) processes cannot be practically applied due to poor pseudoelasticity and mechanical properties. Therefore, the present invention relates to the use of other unique fabrication methods such as capsule-free hot isostatic pressing (CF-HIP) techniques to form new porous shape memory materials with superior mechanical properties especially in relation to pseudoelasticity. Porous shape memory materials such as porous NiTi alloys have been fabricated with adjustable pore distribution, pore size and pores shape by the use of the aforementioned methods. Additionally, the porous shape memory materials can exhibit almost complete pseudoelasticity and superior mechanical properties at austenite finish temperature such as at 37° C. for medical application.

### BRIEF DESCRIPTION OF THE DRAWINGS

Some embodiments of the invention will now be described by way of example and with reference to the accompanying drawings, in which:

FIG. 1 shows optical micrographs of (a) radial section; (b) axial section and (c) radial section (higher magnification) of the porous NiTi shape memory alloys produced according to an embodiment of the invention,

FIG. 2 shows the pore size distributions of the porous NiTi shape memory alloys produced by embodiments of the invention,

FIG. 3 shows optical micrographs of porous NiTi SMAs with different pore characteristics prepared by embodiments of the invention,

FIG. 4 shows the stress-strain curves of the porous NiTi shape memory alloys fabricated according to an embodiment of the invention,

FIG. 5 shows the stress-strain curves of the porous NiTi shape memory alloys fabricated according to another embodiment of the invention,

FIG. 6 shows the morphologies of fractographies of porous NiTi SMAs fabricated by embodiments of the invention after compression tests, and

FIG. 7 shows the internal friction and elastic modulus of porous NiTi SMAs prepared by embodiments of the invention.

### DESCRIPTION OF PREFERRED EMBODIMENTS OF THE INVENTION

For the purposes of promoting an understanding of the principles of porous shape memory materials, such as Ti-50.8 at. % Ni alloy and Ti-30 at. % Ni-20 at. % Cu alloy, fabricated by capsule-free hot isostatic pressing techniques (CF-HIP), the following preferred embodiments of the invention will be described by way of example.

TABLE 1
indicates the porosity of the samples before and after CF-HIP treatment.

| | Theoretical | | Porosity, | Open-pore |
|---|---|---|---|---|
| | density, g/cm³ | Density, g/cm³ | % | ratio, % |
| Compacted | 6.19 | 3.92 | 36.1 | / |
| powder sample | | | | |
| CF-HIP sample | 6.45 | 3.95 | 39.2 | 60.6 |

From this it can be seen that the porosity of the sample after CF-HIP is much higher when compared to the untreated sample. The measured open-pore ratio of the sample reaches at 60.6%, which can be determined by the liquid weighing method.

The preparation process for the sample is described as follows but is not limited to this method. Ni powder with a purity of 99.8% and size of 4-7 μm (Goodfellow Company) and Ti powder with a purity of 99.9% and size of 50-75 μm (Shanghai Reagent Corporation) were used. The powder mixture with the composition of Ti-50.8 at % Ni was blended in a UBM-4 mill (MASUDA Company) for 4 hours. The rotation speed of the mill was 150 rpm and the weight ratio of ball to powder is 4:1. The blended powder mixtures were pressed to cylindrical green samples at a pressure of 100 MPa (mold pressure) using a hydraulic press. The reactive sintering of the green sample was performed at 1050° C. under hot isostatic press in the furnace (ABB Autoclave Systems INC) with 150 MPa (hot pressure), as shown in Table 2. The specimens obtained by CF-HIP were subjected to ageing treatment at 450° C. in a tube furnace under the protection of high purity argon gas for 0.5 h followed by ice-water quenching. The parameters used in this fabrication are not only limited to the parameters as shown in Table 2 below.

TABLE 2
Fabrication and treatment parameters of capsule-free
hot isostatic pressing

| | | Sintering | | | Ageing | |
|---|---|---|---|---|---|---|
| | Mold | tem- | Hot | Sintering | tem- | Ageing |
| | pressure | perature | pressure | time | perature | time |
| | (MPa) | (° C.) | (MPa) | (Hour) | (° C.) | (Hour) |
| Porous | 1-600 | 750-1250 | 1-200 | 0.5-20 | 200-800 | 0.1-100 |
| NiTi | | | | | | |
| alloys | | | | | | |

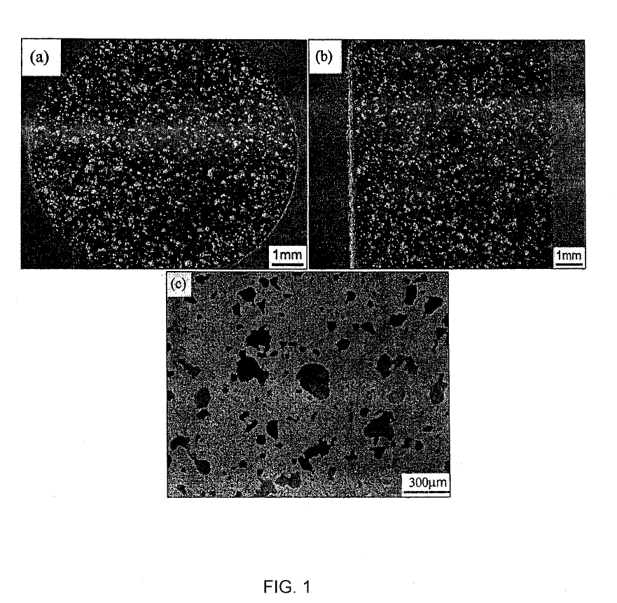

FIG. 1 shows optical micrographs of the porous NiTi alloy fabricated by CF-HIP using an optical microscope (OLYMPUS BH-2). No apparent difference of pore distribution can be observed along radial and axial directions. Moreover, FIG. 1 shows that the distribution of pores was uniform and almost all of the pores were nearly round in shape. The sample fabricated by CF-HIP therefore exhibited good isotropic pore shape and distribution.

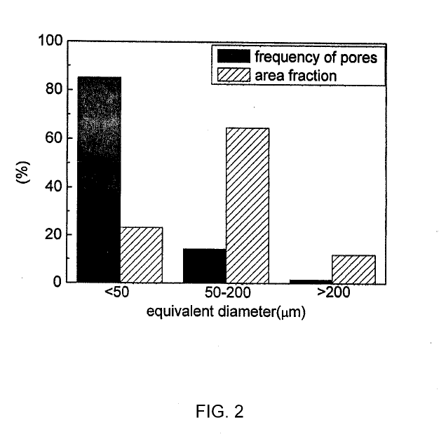

FIG. 2 indicates pore size distribution of the porous NiTi SMAs produced by CF-HIP. The result is the average value obtained from five images randomly taken on different areas of the specimens. Each image was analyzed using a LEICA Microsystem Imaging Solutions (LEICA Qwin Standard). The frequency fraction represents the ratio of the numbers of pores to the total pore numbers in different pore size range, while the area fraction is the ratio of pore area in different range of pore size to the total pore area. It was observed that a number of pores with the size less than 50 μm were dominant. However, the area fraction of the pores was dominated by pores ranging from 50 to 200 μm. Therefore, most of the pore size is ranged from 50 to 200 μm.

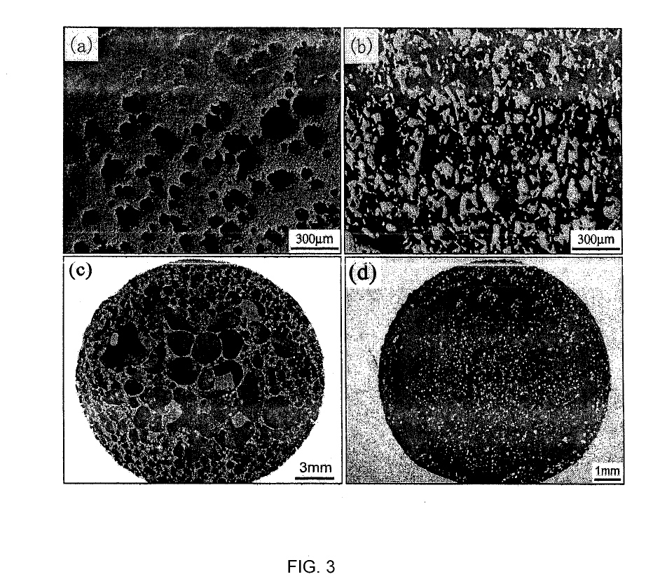

FIG. 3 shows the porous NiTi SMAs with different pore characteristics prepared by CF-HIP. The processing parameters adopted in fabricating the samples are as shown in Table 3 below but not limited to these parameters.

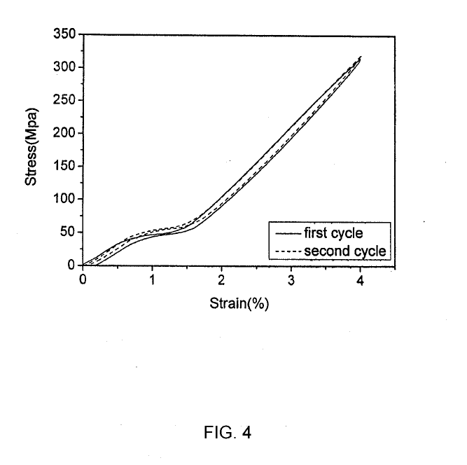

FIG. 4 shows the compression test results of the porous NiTi SMAs prepared by CF-HIP. The sample was sintered by CF-HIP at 1050° C. for 3 hours under 100 Mpa hot pressure. The testing sample was aged at 450° C. for half an hour, and followed by ice-water quenching. The sample was machined into a cylindrical shape with a length of 12 mm and a diameter of 6 mm to characterize superelasticity. The compression test was performed with an Instron 4206 Material Test System at an initial strain rate of 3.33×10⁻³/s. The test was performed at the temperature of human body (38□).The sample showed an incomplete pseudoelasticity in the first cycle. However, in the subsequent cyclic loading, the remaining strain was only about 0.1% after unloading. This indicates that these materials have superior pseudoelasticity even when the deformation is up to 4% strain.

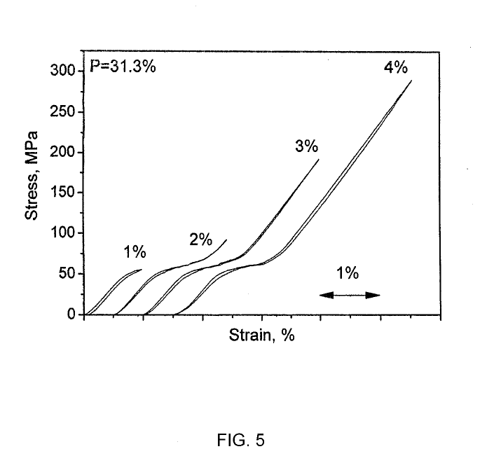

FIG. 5 shows the stress-strain curves of the porous NiTi SMAs prepared by CF-HIP according to embodiments of the invention under various pre-strains. The samples were fabricated by CF-HIP by sintering at 1050° C. for 3 hours under 100 Mpa hot pressure. The samples were aged at 450° C. for half an hour, followed quenching in ice-water. The porosity of the sample was 31.3%, which was denoted top left corner in the figure. The samples under different pre-strains exhibited almost complete pseudoelasticity. This indicated that these materials have complete pseudoelasticity from 1% to 4%, or even higher.

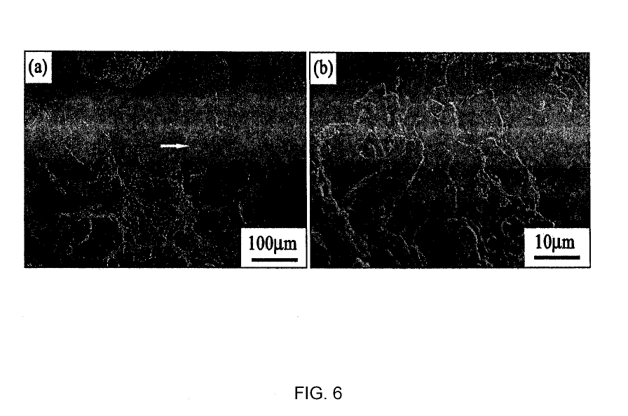

FIG. 6 shows the morphology of fractographies of porous TiNi SMAs fabricated according to embodiments of the invention by CF-HIP after compression tests using a scanning electron microscope (SEM) JEOL-820. The samples were formed by CF-HIP by sintering at 1050° C. for 3 hours under 150 Mpa hot pressure, and aged at 450° C. for 0.5 hours before quenching in iced water. Most of the pores have a spherical shape, and the cracks, indicated by a white arrow in the figure, were found to appear at smooth pore wall. This demonstartes that the local stress concentration is not severe, and there is no sharp angle along the pore wall for crack to initiate preferentially. In addition, some dimples were also observed in the fractography, as shown in FIG. 6(b). This indicated that the porous TiNi SMAs fabricated by CF-HIP breaks in a typical ductile fracture mode, and accordingly has high compressive strength.

TABLE 3
Fabrication parameters of porous NiTi SMAs given in FIG. 3

| | | Sintering | Hot | Sintering | |
|---|---|---|---|---|---|
| | Mold pressure | temperature | pressure | time | |
| | (MPa) | (° C.) | (MPa) | (Hour) | Vesicant |
| (a) | 100 | 1050 | 130 | 5 | No |
| (b) | 100 | 950 | 1 | 3 | No |
| (c) | 100 | 1050 | 150 | 3 | Yes |
| | | | | | 1 wt. % TiH₂ |
| (d) | 400 | 1050 | 150 | 3 | No |

It was noted that 1 wt. % Ti powder was replaced by TiH₂ powder only in the fabrication of the sample in FIG. 4(c). The parameters mentioned are not only limited to the parameters. Not only pore shape can be controlled by changing process parameters, but also pore size can be adjusted. Pore shape can be near spherical shape, also can be polygonal shape. Moreover, pore distribution of the porous NiTi SMAs can be controlled, such as small pores at outside core and big pores at inside core; or porous at outside core, dense at middle core and porous in the center.

Table 4 below shows the pore characteristic parameters of porous NiTi SMAs shown in FIG. 3.

TABLE 4

| Pore | | Open | Open-pore | Pore diameter, |
|---|---|---|---|---|
| characteristic | Porosity, % | porosity, % | ratio, % | μm |
| (a) | 27.13 | 11.76 | 43.35 | 50-200 |
| (b) | 43.06 | 36.03 | 83.69 | <50 |
| (c) | 77.59 | 6.46 | 8.32 | 300-3000 |
| (d) | 31.6 | 16.5 | 52.1 | 50-200 |

It can be seen that porosity can be adjusted at a wide range, such as from 27% to 78%. It can also be seen that pore size also can be controlled, such as from 50 to 3000 μm.

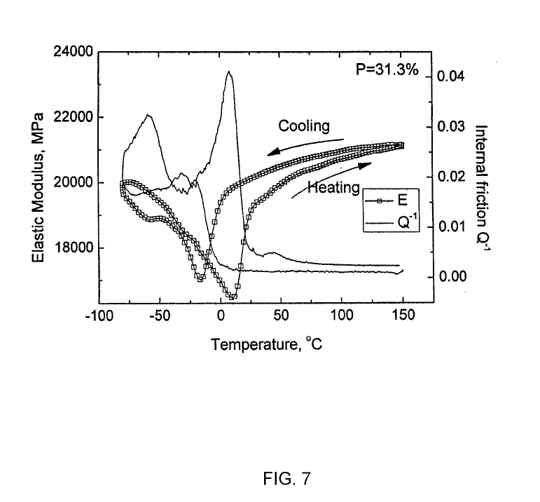

FIG. 7 shows the internal friction (IF) and elastic modulus of porous TiNi SMAs fabricated according to embodiments of the invention by CF-HIP (sintering at 1050° C. for 3 hours under 150 Mpa hot pressure, and aged at 450° C. for 0.5 hours followed by quenching in iced-water), which were measured using a dynamic mechanical analyzer (DMA 2980, TA-Instruments) in a temperature range from −80° C. to 150° C. with heating/cooling rate of 5° C./min. The DMA equipment was set in multi-frequency testing mode and single cantilever (clamp section). The specimens were cut by electrical discharge machining, with geometry of 30×4×1.2 (length×width×thickness, mm). The strain amplitude used in this study was 1.33×10⁻⁴. It can be seen that the sample has a high IF peak about 4% at 10° C. during heating, and the IF value at low temperature, viz. in martensite phase, reached as high as about 2%. As known, the damping performance is the capability of a material to absorb the vibration energy, which can be characterized by IF. This indicated that these materials have good damping property.

While the invention has been illustrated and described in detail in the drawings and foregoing description, the same is to be considered as illustrative and not restrictive in character, it is reasonable to think that only the preferred embodiments have been shown and described and that all changes and modifications that come within the spirit of the invention are desired to be protected.

We claim the following:

1. A porous shape memory material having a pseudoelasticity of 0.1% to 50%.

2. A material as claimed in claim 1 wherein said material is a nickel-titanium alloy.

3. A material as claimed in claim 2 wherein the alloy includes at least one further component.

4. A material as claimed in claim 3 wherein said at least one further component comprises palladium or vanadium.

5. A material as claimed in claim 2 wherein said at least one further component comprise(s) less than 30% of the total weight of said material.

6. A material as claimed in claim 1 wherein said material is fabricated by a method selected from the following:
(a) controlled hot isostatic pressing
(b) capsule-free hot isostatic pressing
(c) powder metallurgies
(d) foaming by gas injection
(e) foaming with blowing agent
(f) vapour deposition
(g) electro-deposition technique
(h) any combination of (a) to (g).

7. A material as claimed in claim 1 having a porosity of between 1% and 99%.

8. A material as claimed in claim 1 having a pore size of between 50 μm to 5000 μm.

9. A material as claimed in claim 1 wherein the pore distribution can be adjusted by selecting fabrication parameters.

10. A material as claimed in claim 1 wherein said material has an isotropic pore distribution in axial and radial directions, and a homogenous distribution in each direction.

11. A material as claimed in claim 1 wherein the pore size and pore distribution vary in a radial direction can be controlled.

12. A material as claimed in claim 11 wherein a said material is dense at a radially outer location and porous at a radially inner location.

13. A material as claimed in claim 11 wherein said material is dense at a radially inner location and porous at a radially outer location.

14. A material as claimed in claim 11 wherein said material is porous at radially outer and inner locations and dense at an intermediate location therebetween.

15. A material as claimed in claim 1 wherein the pore shape can be adjusted to different shapes such as a spherical or polygonal shape.

16. A material as claimed in claim 1 wherein the said material has low local stress concentration around the pores.

17. A material as claimed in claim 1 wherein the pores can be interconnected or not interconnected.

18. A material as claimed in claim 1 having a Young's modulus of from 0.1 GPa to 50 GPa.

19. A material as claimed in claim 1 having a yield strength of from 1 MPa to 500 MPa.

20. A material as claimed in claim 1 wherein the damping properties of the material are in the range from 0.1% to 9%.

21. A material as claimed in claim 1 wherein the austenite start and finish transformation temperatures that lead to said pseudoelasticity can be controlled by ageing the said material at a temperature of from 200° C. to 1000° C.

22. A material as claimed in claim 1 wherein the austenite start and finish transformation temperatures that lead to said pseudoelasticity can be controlled by ageing said material for a time from 15 minutes to 24 hours.

23. A material as claimed in claim 1 wherein the austenite start and finish transformation temperatures that lead to said pseudoelasticity can be controlled by various cooling methods including but not limited to water quenching, air quenching and furnace cooling.

24. A material as claimed in claim 1 wherein the martensite start and finish transformation temperatures that lead to a shape memory effect can be controlled by ageing the said material at a temperature of between of 200° C. to 1000° C.

25. A material as claimed in claim 1 wherein the martensite start and finish transformation temperatures that lead to a shape memory effect can be controlled by ageing the said material for a time from 15 minutes to 24 hours.

26. A material as claimed in claim 1 wherein the martensite start and finish transformation temperatures that lead to a shape memory effect can be controlled by various cooling methods including but limited to water quenching, air quenching and furnace cooling.

27. A material as claimed in claim 1 wherein the material exhibits pseudoelasticity at human body temperature.

28. An orthopedic implant made of a material as claimed in claim 1.

29. A device for joint replacement such as for hip, knee, ankle, shoulder, elbow, wrist and finger made of a material as claimed in claim 1.

30. An intervertebral disc prosthesis made of a material as claimed in claim 1.

31. A vascular implant made of a material of claim 1.

32. An esophageal implant made of a material of claim 1.

33. A material as claimed in claim 1, wherein the material is an engineering materials used for energy absorption.

34. A passive damping device made of a material as claimed in claim 1.

35. A method of forming a porous shape memory material comprising sintering an alloy material at high temperature and under isostatic pressure.

36. A method as claimed in claim 35 wherein said alloy is a Ni—Ti alloy.

37. A method as claimed in claim 35 wherein said sintering is carried out at a temperature of between 750° C. and 1250° C.

38. A method as claimed in claim 35 wherein said sintering is performed for 0.5 to 20 hours.

39. A method as claimed in claim 35 wherein said isostatic pressure is in the range of 1 to 200 Mpa.

40. A method as claimed in claim 35 wherein after sintering said material is aged at between 200° C. to 800° C.

41. A method as claimed in claim 40 wherein said ageing is performed for 0.1 to 100 hours.

42. A method as claimed in claim 40 wherein said material is quenched in iced water after said ageing.
